(19) United States
(12) Patent Application Publication
(10) Pub. No.: US 2025/0147000 A1
(43) Pub. Date: May 8, 2025

(54) METHOD AND SYSTEM FOR SENSING SOIL PROPERTIES AND DEPLOYING PREDICTIVE ANALYTICS FOR SOIL PROPERTIES

(71) Applicant: Michael Murray, Lexington, MA (US)

(72) Inventor: Michael Murray, Lexington, MA (US)

(21) Appl. No.: 19/008,132

(22) Filed: Jan. 2, 2025

Related U.S. Application Data
(63) Continuation-in-part of application No. 17/378,370, filed on Jul. 16, 2021, now abandoned.

(51) Int. Cl. G01N 33/24; G06F 3/01

(52) U.S. Cl. CPC G01N 33/246; G01N 33/245; G06F 3/011

(57) ABSTRACT

A system for predicting ideal environmental conditions to grow plants establishing a connection with at least one sensor, wherein the sensor is able to collect data related to at least one environmental condition; grouping, by one or more processors, the at least one sensor into at least one zone; calibrating the at least one sensor within each zone, wherein the calibration is based on a set of environmental conditions desired for a plant; collecting data related to the at least one environmental condition from the at least one sensor; manipulating the collected data, wherein the data is manipulated from a first format to a second format, wherein the second format is readable by a predictive analyzing engine; performing a predictive model manipulation on the manipulated collected data; alerting a user with a recommendation based on the predictive model; and manipulating a user interface to identify specific environmental conditions.

### CROSS-REFERENCE TO RELATED APPLICATIONS

This application claims the benefit of priority under 35 USC 120 of U.S. application Ser. No. 17/378,370 filed Jul. 16, 2021. The disclosure of the prior applications is considered part of (and is incorporated by reference in) the disclosure of this application.

### BACKGROUND

This disclosure relates generally to a system for monitoring soil properties, and in particular to the method for collecting soil properties and predicting soil requirements to maintain the ideal environment for the plants.

The monitoring of the moisture of soil for the purpose of optimizing the growth of crops has become increasingly important today, particularly in the environment of large, corporate farming operations. There are two common practices associated with the installation of soil moisture probes in the soil. The most prevalent method involves the installation of a soil monitoring probe in the ground once the plant emerges after planting (actually a series of probes to cover an entire planted field). Each probe is then connected to a telemetry system that provides power and receives the measured data from the probe. The telemetry will regularly upload the received data to a central database using cellular or other wireless technology.

Generally, soil moisture is the water that is trapped in the spaces among soil particles. Determining the amount of soil moisture has important implications for a number of reasons, especially in the science of agriculture. For example, soil moisture serves as a solvent and carrier of food nutrients for plant growth, as well as regulates soil temperature. As such, crop yield is often determined by the amount of water available in the soil rather than the deficiency of other food nutrients.

A less common practice is to install the probe(s) in the soil and then trench the connecting cable to the perimeter of the field (typically about 100 meters away). This will allow the probe to reside in the field continuously for several years, providing data to the grower over the entire year. There are several drawbacks with the trenching method. First, it is a cumbersome and expensive exercise to trench the cable (to each probe). Second, there are several cases where normal field operations will result in one or more of the cables being severed, thereby breaking the connection to the probe.

While soil moisture sensing systems already exist, they all suffer from certain flaws in one way or another due to their measurement technique. Examples of known techniques for measuring soil moisture include: receive signal strength indicator (RSSI)/radio attenuation and time-domain reflectometry (TDR). (This is not an exhaustive list.)

These sensing systems utilizing the above techniques tend to be highly sensitive to the makeup of the soil directly next to its sensors, and provide measurements that can be highly variable due to issues in installation or other sources. The volume of soil measured may also be an issue, as each of the sensing systems described above measures only a very short distance from its sensor probes (generally just a few centimeters).

What is needed is a system and method that permits the probe to reside continuously in the field without the need for expensive trenching, and without the risk of damage to the equipment due to normal field operations. It is believed that a wireless probe transmission system that is buried in close proximity to each probe, is the solution to this problem.

The operator thus has the task of monitoring and controlling a variety of system parameters to achieve the best conditions for the specific plants in the installation. This can be time consuming. A failure to properly control the parameters may result in plant harm and financial loss. Additionally, if a component failure occurs while the operator is not on site, it may be detected too late to prevent harm. Component failures such as leaks, failed pumps, faulty temperature control devices or faulty lamps can occur at any time.

Often, it is financially advantageous to ensure the plants are growing at the fastest rate possible using the least amount of resources. This often requires detailed analysis of present and historical data, looking for trends between nutrient and environmental conditions and plant response. This requires keeping accurate measurements of measured conditions and a method of recording plant growth and behavior, typically over the course of one or more growing seasons. Conventionally this is done by keeping records by hand, and requires additional time and effort, with the possibility of mistakes.

Therefore, it is desired for a system to monitor the plants and soil conditions, collect the data, and provide the data in an easy affordable and manageable way.

### SUMMARY

In a first embodiment, the present invention is a computer implemented method for predicting ideal environmental conditions to grow plants, the method comprising: establishing, by one or more processors, a connection with at least one sensor, wherein the sensor is able to collect data related to at least one environmental condition; grouping, by one or more processors, the at least one sensor into at least one zone; calibrating, by the one or more processors, the at least one sensor within each zone, wherein the calibration is based on a set of environmental conditions desired for a plant and each of the at least one sensor within the zone are calibrated substantially similar; collecting, by the one or more processors, data related to the at least one environmental condition from the at least one sensor; manipulating, by the one or more processors, the collected data, wherein the data is manipulated from a first format to a second format, wherein the second format is readable by a predictive analyzing engine; performing, by the one or more processors, a predictive model manipulation on the manipulated collected data; alerting, by the one or more processors, a user with a recommendation based on the predictive model; and manipulating, by the one or more processors, a user interface to identify specific environmental conditions to be adjusted and a predetermined adjustment of those environmental conditions.

In a second embodiment, the present invention is a system for predicting ideal environmental conditions to grow plants, the system comprising: a CPU, a computer readable memory and a computer non-transitory readable storage medium associated with a computing device; establishing a connection with a group of sensors and at least on environmental system; forming zones, wherein each zone contains at least one sensor; setting a set of parameters for a first group of sensors within one of the zones; collecting environmental data from the first group of sensors based on the set of parameters,, wherein the environmental data is received by a gateway; manipulating the collected environmental data, wherein the data is manipulated from a first format to a second format, wherein the second format is readable by a predictive analyzing engine; performing a predictive model manipulation on the manipulated collected environmental data, wherein a future value of the environmental data is calculated; generating visualizations of the collected environmental data and the future value of the environmental data; and presenting the visualizations to a user's interface of a device.

In a third embodiment, the present invention is a computer program product for predicting ideal environmental conditions to grow plants, the computer program product comprising: a computer non-transitory readable storage medium having program instructions embodied therewith, the program instructions executable by a computing device to cause the computing device to: program instructions to calibrate a set of sensors, wherein a frequency of at least one circuit is adjusted based on a desired environmental condition to be measured; program instructions to collect data from the set of sensors, wherein the collected data is related to a set of variables and is provided to a gateway, and wherein the data is collected at a predetermined time frames; program instructions to process the collected data; program instructions to manipulate the collected data from a first format to a second readable format for a predictive engine; program instructions to analyze the manipulated collected data, wherein the manipulated collected data is analyzed to determine future values a set of parameters based on a set of threshold values; and program instructions to generate visualizations of the collected data and the future values.

### BRIEF DESCRIPTION OF THE DRAWINGS

FIG. 1 depicts a distributed network environment, in accordance with one embodiment of the present invention.

FIG. 2 depicts a block diagram depicting the internal and external components of the server and computing devices of FIG. 1, in accordance with one embodiment of the present invention.

FIG. 3 depicts an illustration of the distributed networked environment, in accordance with one embodiment of the present invention.

FIG. 4 depicts a flowchart of the operational steps to construct the distributed network environment and collect data, in accordance with one embodiment of the present invention.

FIG. 5 depicts a sensor, according to an embodiment of the present invention.

FIG. 6 depicts a sensor, according to another embodiment of the present invention.

FIG. 7 depicts the sensor's computing elements, according to an embodiment of the present invention.

FIG. 8 depicts a user interface showing the collected data of the sensor, according to an embodiment of the present invention.

FIG. 9 depicts a user interface showing the setting up of zones, according to an embodiment of the present invention.

FIG. 10 depicts an illustration of a virtual reality user interface, according to an embodiment of the present invention.

FIG. 11 depicts an illustration of an augmented reality user interface, according to an embodiment of the present invention.

### DETAILED DESCRIPTION

The preferred embodiments of the present invention, as well as other embodiments thereof, will now be further described with reference to the accompanying drawings, wherein like reference numerals designate like or corresponding parts throughout the several views. Although the invention will be illustratively described hereinafter and is shown in the drawings mostly with reference to residential construction, it should be understood that the invention is not limited to residential environments, but could be utilized in the construction of other single- and multi-story structures, including, but not limited to, office, retail, industrial, place of assembly, educational and laboratory structures.

As required, detailed embodiments of the present invention are disclosed herein; however, it is to be understood that the disclosed embodiments are merely exemplary of the invention that may be embodied in various forms. The figures are not necessarily to scale, and some features may be exaggerated to show details of particular components. Therefore, specific structural and functional details disclosed herein are not to be interpreted as limiting, but merely as a basis for the claims and as a representative basis for teaching one skilled in the art to variously employ the present invention.

As mentioned above, traditional soil moisture sensing systems have a number of limitations due to the underlying principles and techniques upon which the sensors operate. Disclosed herein is a novel system and method for detecting and measuring soil moisture content that addresses a number of key deficiencies of traditional soil moisture detection systems. According to exemplary embodiments of the present system and method, the effects of a magnetic field, or an “H” field, are used to accurately measure soil moisture content at greater depths, with greater measurement radii, across different soil types, and at different levels of depth.

Disclosed herein is a method and system for monitoring and predicting the soil properties through the use of in ground sensors which are connected to a network, which is able to collect the sensor data, deploy predictive analytics and identify information which would be useful to a user to properly maintain the soil.

Those skilled in the art will recognize many modifications may be made to this exemplary environment without departing from the scope of the present disclosure. For example, it will be appreciated that aspects of the present disclosure are not dependent upon data structure formats, communications protocols, file types, operating systems, database management system, or peripheral device specifics. Accordingly, the following description is provided without reference to specific operating systems, protocols, or formats, with the understanding that one skilled in the art will readily be able to apply this disclosure to a system and format of choice.

Still further, as used herein, “interface” is intended to include data structures, virtual and physical connections between devices, computer-human user interface, and other mechanisms that facilitate the exchange of data between computer systems and/or control of one or more such systems. In one embodiment, an interface requires a minimum or no user data entry or manual delivery of data from one system to another. In another embodiment, data that needs to be entered manually may be retained and reused within the system, reducing future data entry requirements.

According to the present disclosure, a user interacts with a computer system and controls provided thereby to design a structure. In the process, the system may communicate with other systems to obtain data, verify data, deliver data, store or retrieve data, etc. Those other systems may be interfaces to other computer-user interactions or be autonomous or some combination of the two. By way of a network, the systems and methods thereby facilitate collaboration between multiple individuals and/or organizations in the design, analysis, and implementation of a structure.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods, and computer program products according to various embodiments of the present invention. In this regard, each block in the flowcharts may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the flowchart illustrations, and combinations of blocks in the flowchart illustrations, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

### Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

### Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

### Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

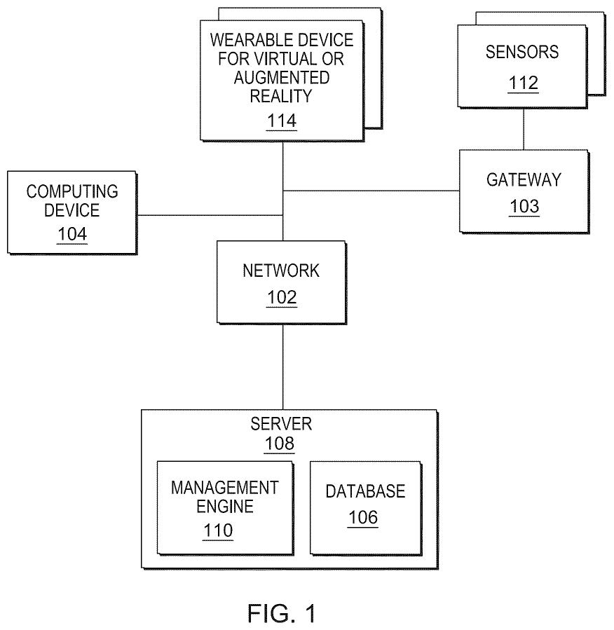

FIG. 1 depicts a block diagram of a computing environment 10 in accordance with one embodiment of the present invention. FIG. 1 provides an illustration of one embodiment and does not imply any limitations regarding the environment in which different embodiments maybe implemented.

In the depicted embodiment, computing environment 100 includes network 102, computing device 104, database 106, server 108, sensors 112, and wearable device for virtual reality and and/or augmented reality equipment 114. Computing environment 100 may include additional servers, computers, or other devices not shown. A distributed network environment 100 is shown, comprising hardware and software, within which various embodiments of the present disclosure may be employed. More specifically, distributed network environment 100 comprises multiple interconnected elements of hardware, each running software, allowing those elements of hardware to communicate with one another, whether by wired or wireless connection.

Network 102 may be a local area network (LAN), a wide area network (WAN) such as the Internet, any combination thereof, or any combination of connections and protocols that can support communications between computing devices 104, gateway 103, server 108, sensors 112, and wearable device for virtual reality and and/or augmented reality equipment 114 in accordance with embodiments of the invention. Network 102 may include wired, wireless, or fiber optic connections. Alternatives to computing devices 104, or additional computer mechanisms include personal computers, servers that are personal computers, minicomputers, personal digital assistants (PDAs), mainframes, etc. The network within which the various embodiments of the present disclosure operates may also comprise additional or fewer devices without affecting the scope of the present disclosure.

Gateway 103 may be a management server, a web server, or any other electronic device or computing system capable of processing program instructions and receiving and sending data. The gateway functions to translate the information received from the sensors 112 to the network 102.

Computing device 104 may be a management server, a web server, or any other electronic device or computing system capable of processing program instructions and receiving and sending data. The computing device 104 receives the data directly from sensors 112 and transmits that data to the server 108. The processed data is then sent back to the computing device 104 for the user to view and use to make changes during the care of the plants. In some embodiments, computing device 104 may be a laptop computer, tablet computer, netbook computer, personal computer (PC), a desktop computer, or any programmable electronic device capable of communicating with gateway 103, server 108, sensors 112, and wearable device for virtual reality and and/or augmented reality equipment 114 via network 102. In other embodiments, computing device 104 may represent a server computing system utilizing multiple computers as a server system, such as in a cloud computing environment. In another embodiment, computing device 104 represents a computing system utilizing clustered computers and components to act as a single pool of seamless resources. computing device 104 may include components, as depicted and described in further detail with respect to FIG. 1.

In some embodiments, the computer device 104 needs to be within a predetermined proximity to the sensors 112. This is based on the internal components of sensors 112 and their ability to wirelessly transmit data.

Database 106 may be a repository that may be written to and/or read by design engine 110. In one embodiment, database 106 is a database management system (DBMS) used to allow the definition, creation, querying, update, and administration of a database(s). In the depicted embodiment, database 106 is connected to network 102. In other embodiments, database 106 resides on servers or computing devices, provided that database 106 is accessible to gateway 103, computing device 104, and wearable device for virtual reality and and/or augmented reality equipment 114.

Server 108 may be a management server, a web server, or any other electronic device or computing system capable of processing program instructions and receiving and sending data. In other embodiments server 108 may be a laptop computer, tablet computer, netbook computer, personal computer (PC), a desktop computer, or any programmable electronic device capable of communicating via network 102. In one embodiment, server 108 may be a server computing system utilizing multiple computers as a server system, such as in a cloud computing environment. In one embodiment, server 108 represents a computing system utilizing clustered computers and components to act as a single pool of seamless resources. In the depicted embodiment management engine 110 and database 106 are located on server 108. Server 108 may include components, as depicted and described in further detail with respect to FIG. 1.

Management Engine 110 operates to perform the analysis on the data collected form the sensors 112 and also to perform the predictive analysis on the future soil conditions and environment conditions based on previously collected data as well as create and present the augmented reality and/or virtual reality to the wearable device 114. The management engine 110 is able to process the collected data to generate information to the computing device which is based on the user's requests. The management engine 110 is also able to generate predictive data related to potential future conditions of the soil or the environment to assist the user in anticipating issues or changes in conditions of the soil. In the depicted embodiment, management engine 110 utilizes network 102 to access the computing devices 104, gateway 103, and the wearable device for virtual reality and and/or augmented reality equipment 114. In one embodiment, management engine 110 resides on gateway 103 or computing device 104. In other embodiments, management engine 110 may be located on another server or computing device, provided management engine 110 has access to database 106 and the other components of computing environment 100.

Sensors 112 provides for the collection of data from the soil and is able to wirelessly transmit the data to a gateway 103, network 102, server 108, or computing device 104. The sensors measures soil moisture, salinity, temperature, humidity, sun light, and other measurable values of a region of soil at its ground location and periodically transmits these values to the respective device. The sensors may be connected wirelessly to one another or individually connected to the gateway 103. In some embodiments each sensor has QR codes or readable codes on that that allow a computing device 104 or a wearable device 114 to read the code and access information about the specific sensor. This information can be related to the sensor features, the sensor readings, the plant, the environment, the zone, or other data which can be programed to be associated with each specific sensor.

Wearable device 114 for virtual reality and and/or augmented reality equipment 114 provide the device for the user, operator, worker, or personnel (hereinafter “user”) to experience and see the virtual reality environment or the augmented reality information. Typically, head mounted display systems worn by the user such as headsets or glasses, provide all the necessary components to allow the virtual reality or augmented reality features and aspects of the present invention to be experiences. These devices may include but are not limited to a head-mounted display system including a user interface, user-sensing system, environment-sensing system, and a processor. The processor may be an isolated component separate from the head-mounted system or maybe integrated with one or more components of the head-mounted system or another component of the system. These wearable devices 114 are able to read the QR code or readable costs associated with each sensor, plot, zone, or other aspect of the facility which can be accessed or controlled by the present system.

Additional elements of hardware include, but are not limited to, network appliances such as remote storage, each communicating via the network 102. The computer devices and servers generally may be referred to as computer devices. Other computer devices, such as mobile computationally enabled telephone handsets (so called “smart phones”), tablet-style computer devices, and so on may also form a part of network environment 100.

As is well known, software components supporting computing devices 104, server 108, and additional network appliances, and so on include or reference logic and/or data that may form a part of the software component or be embodied in or retrievable from some other hardware of software device or signal, either local or remote and coupled via a network or other data communications device.

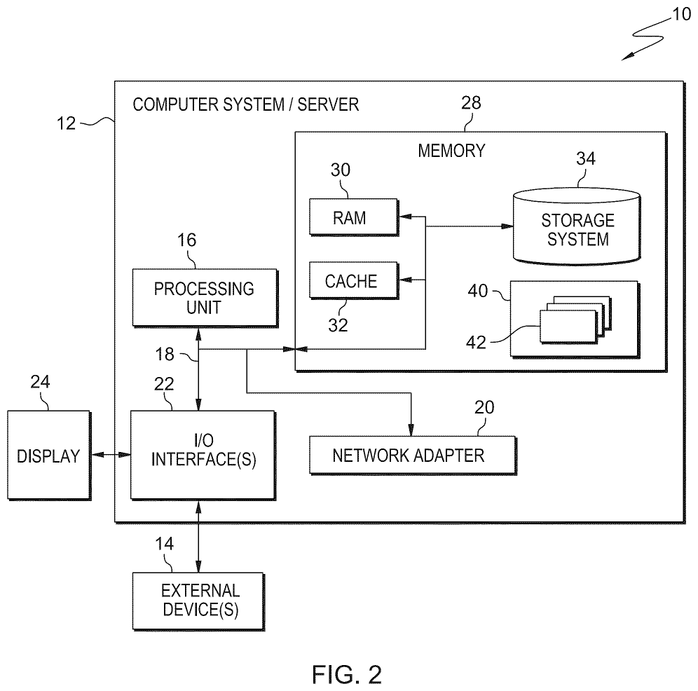

FIG. 2 depicts a block diagram depicting the internal and external components of the server and computing devices of FIG. 1, in accordance with one embodiment of the present invention. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purposes or special purpose computing system environments or configurations. Examples of well-known computing systems, environments, and/or configurations that may be suitable for use with computer system/server 12 include, but are not limited to, personal computer systems, server computer systems, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 2, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random-access memory (RAM) 30 and/or cache memory 32. Computer system/server 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to microcode, device drivers, redundant processing units, external disk drive arrays, RAID systems, tape drives, and data archival storage systems, etc.

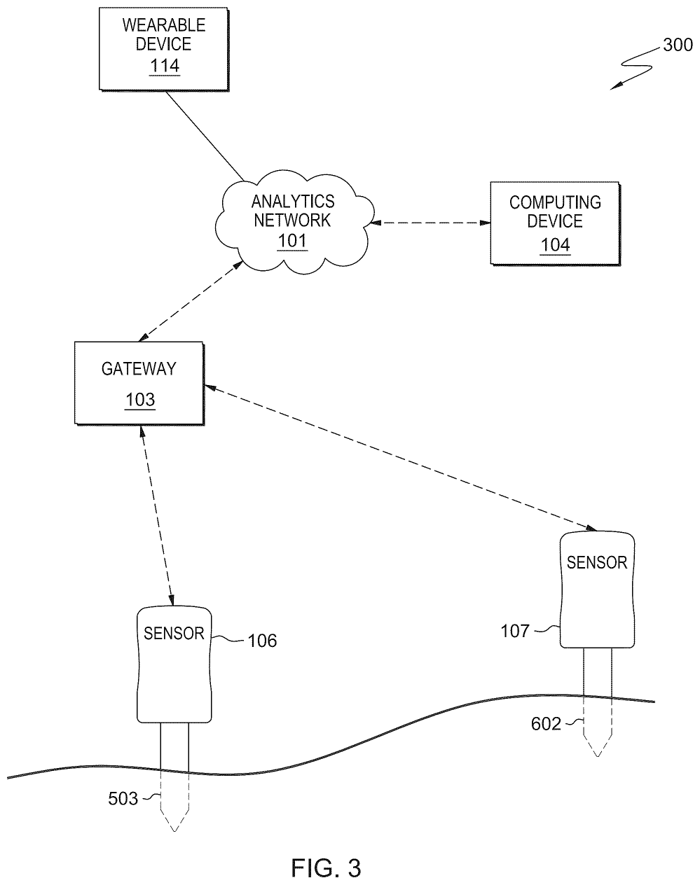

FIG. 3 depicts an illustration of a working environment 300, in accordance with one embodiment of the present invention. The system comprises a set of sensors 112, a gateway 103, an analytic network 101, a computing device 104 and a wearable device 114. The system is consolidated to combine the management engine 110 with the network 104 to form an analytic network 101 which is capable of processing the data received from the gateway 103 and provide the analyzed data directly to the computing device 104 or the wearable device 114. This embodiment employs the use of a cloud analytics design.

Gateway 103 may be a management server, a web server, or any other electronic device or computing system capable of processing program instructions and receiving and sending data. The gateway functions to translate the information received from the sensors 112 to the analytic network 101. Data which is generated from sensors 112 is only applicable to the sensors, and the gateway translates this data to a readable format for either the computing device 104, management engine 110, the wearable device 114, or the database 106. The gateway 103 includes a radio modem capable of communicating with the sensors 112 and the network 102, a network interface capable of communicating with the sensors 112 and the network 102, and a digital controller coupled to the radio modem and to the network interface. The digital controller passes data packets received from the sensors 112 to the computing device 104, management engine 110, or the database 106, and passes data packets received from the computing device 104, management engine 110, or the database 106 to the sensors 112, after performing any necessary translations to the data packets. The digital controller further maintains a map of the links. By maintaining a map of the first network links, the gateway 103 is able to properly address packets received from either the sensors or the computing device 104, management engine 110, or the database 106 to the destination and to maintain and upgrade their data communication paths.

In the depicted embodiment, the gateway 103 is able to communicate with the sensors 112 and provide all this data directly to the computing device 104 or the wearable device 114 regardless of location due to the wireless transmitting properties of the gateway 103. This creates an advantage that the computing device 104 or the wearable device 114 does not need to come within proximity to the sensors 112 and can receive the data regardless of location and positioning relative to the sensors 112. In larger operations this becomes advantageous and provides an increase to the overall efficiency of the system.

The system provide analysis services to users. For example, users may enter an agreement to allow their sensor data to be gathered and customized data analysis services are offered to the customer (e.g., predictive maintenance notifications, real-time system monitoring, automated email alerting services, automated technical support notification, risk assessment, etc.). Alternatively, the user may subscribe to one or more available cloud analytics services, and optionally allow their system data to be maintained. In some embodiments, a user may be given an option to subscribe to cloud analytics services without permitting their data to be stored for collective analysis with data from other systems. In another exemplary agreement, user s may be offered a discount on cloud analytics services in exchange for allowing their system data to be anonymously migrated to BDFM data storage 1102 for collective analysis.

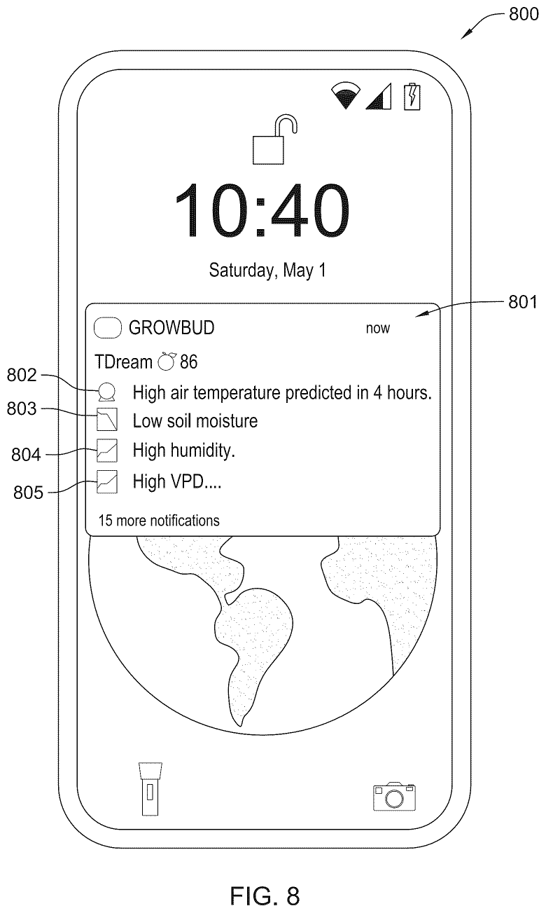

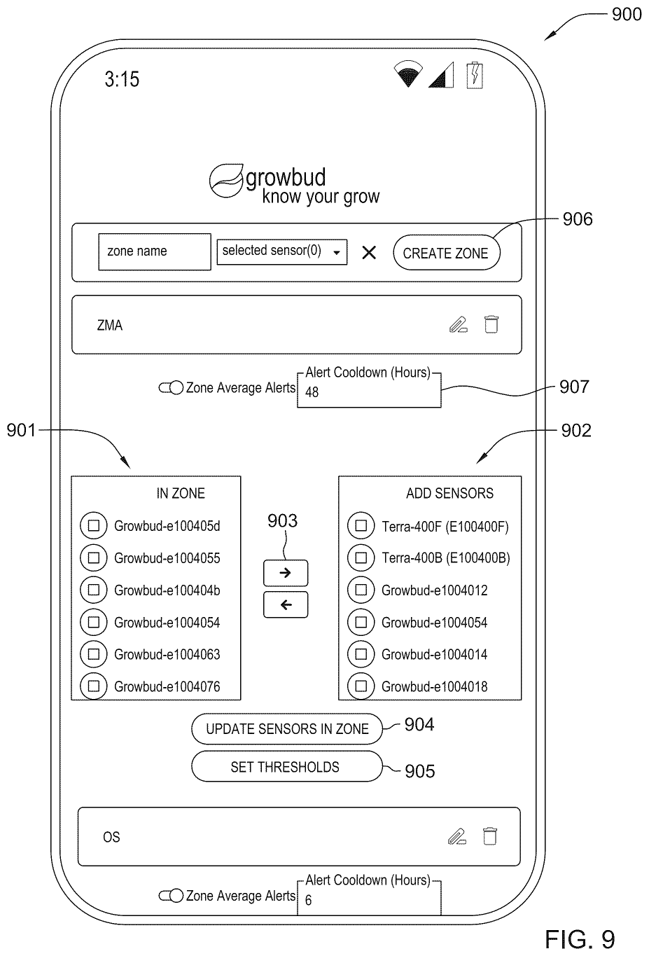

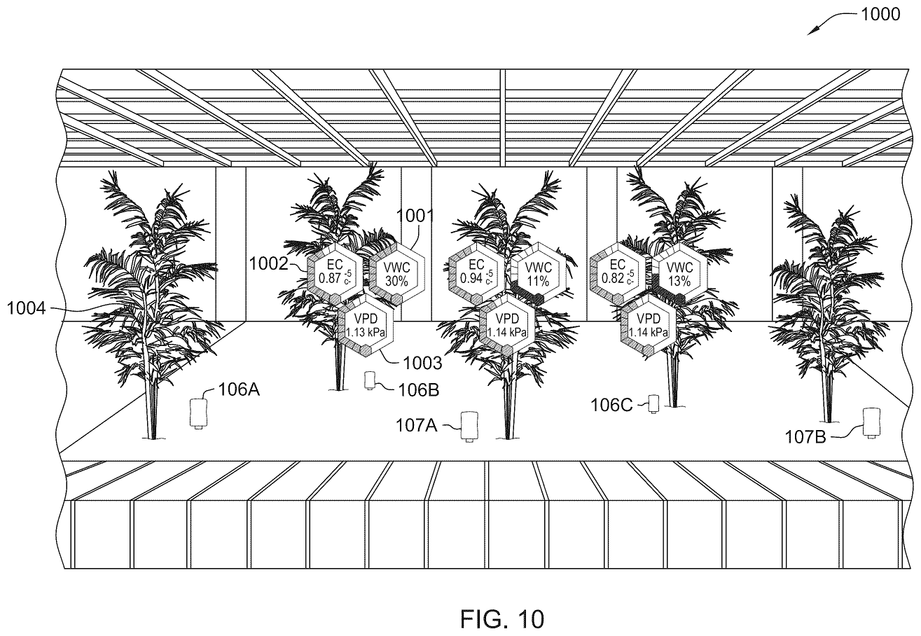

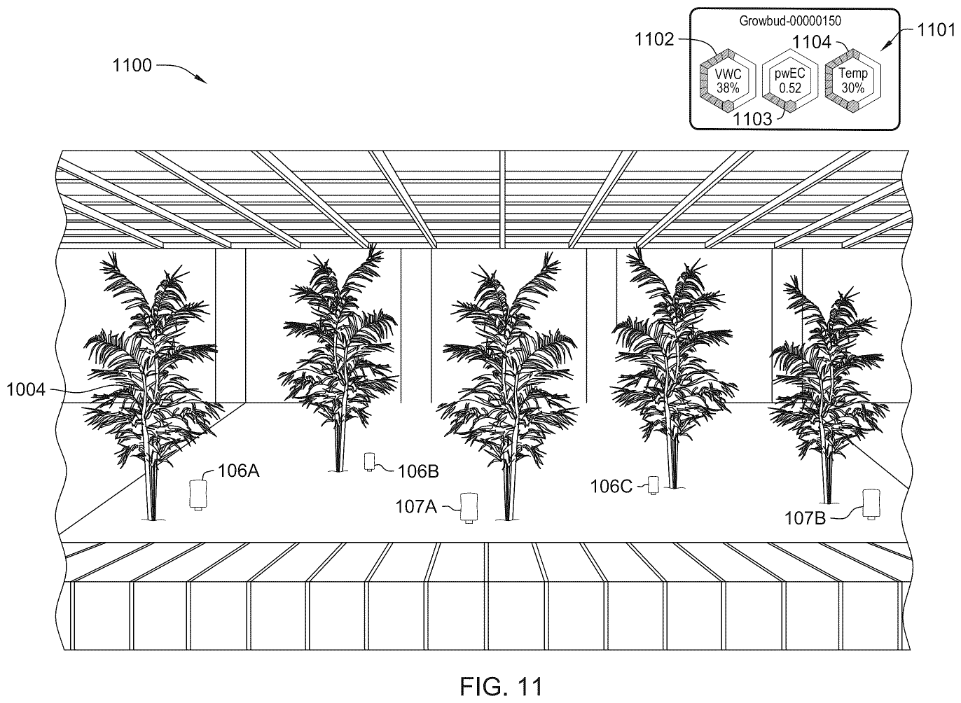

Since the cloud-based analysis system maintains accurate and detailed documentation of each user's devices, setting, environment, and the like, analysis system can generate customized system configuration recommendations based on comparison of the user's system configuration and/or requirements. The system is able to generate a report, or an interactive user interface based on the user specific data and requirements as shown in FIGS. 8 and 9 and also can be used to create the augmented reality shown in FIG. 10 or the virtual reality shown in FIG. 11.

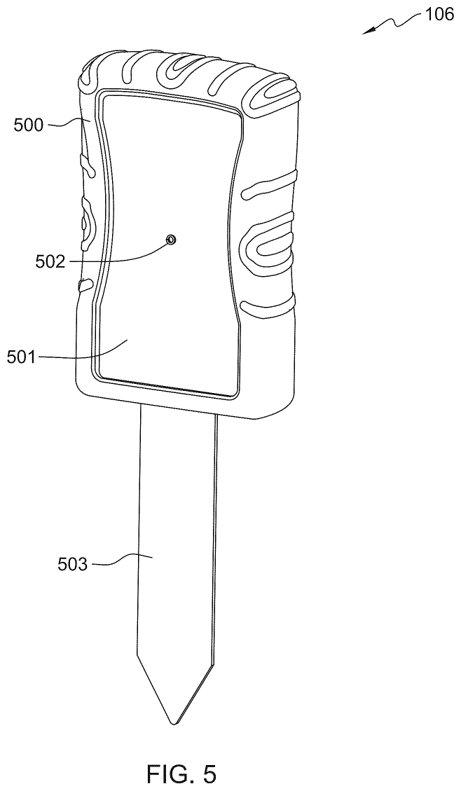

An example of sensor 112, shown in FIG. 5 depicts the sensor 106, according to an embodiment of the present invention. The sensor is comprised of a housing 501, probe 503, and a cover 500. The housing 501 encapsulates a circuit board and a portion of the probe 503. The probe 503 is in physical and electrical communication with the circuit board. The probe 400 contains impedance sensing nodes that are used to collect voltage readings. The cover 500 is a rubber or protective cover designed to protect the housing 502 and the internals from impact. In the depicted embodiment, an example of a code (e.g. a QR code) 502 can be affixed to the sensor for the user with the wearable device 114 to access the sensor's data.

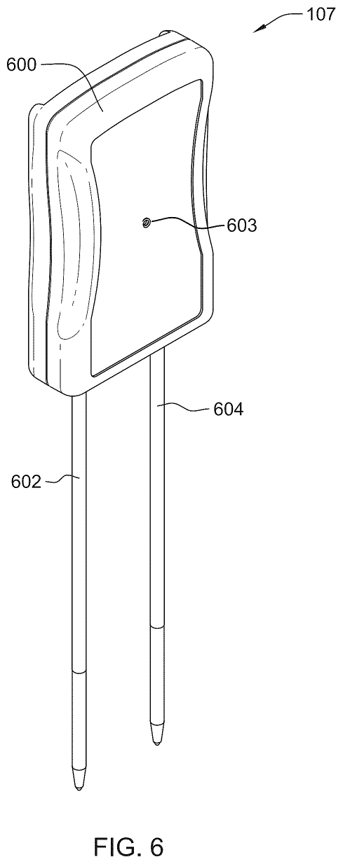

Another example of the sensor 112, shown in FIG. 6 depicts the sensor 107, according to an embodiment of the present invention. The sensor is comprised of a housing 600, probes 602 and 604. The housing 600 encapsulates a circuit board and a portion of the probes 602 and 604. The probes 602 and 604 are in physical and electrical communication with the circuit board. The probes 602 and 604 collect different voltage readings which are supplied to the internal computing device to calculate various properties from the soil. The cover 500 is a rubber or protective cover designed to protect the housing 200 and the internals from impact. In the depicted embodiment, sensor 602 is able to collect environment readings such as sunlight, humidity, rain, and the like. This sensor 107 may also have a QR code or readable code attached to the exterior of the surface.

In some embodiments the sensors 106 and 107 may have Radio Frequency Identification (RFID) chips (or the like) which are able to interact with the wearable device 114 if no QR code is present on the sensor.

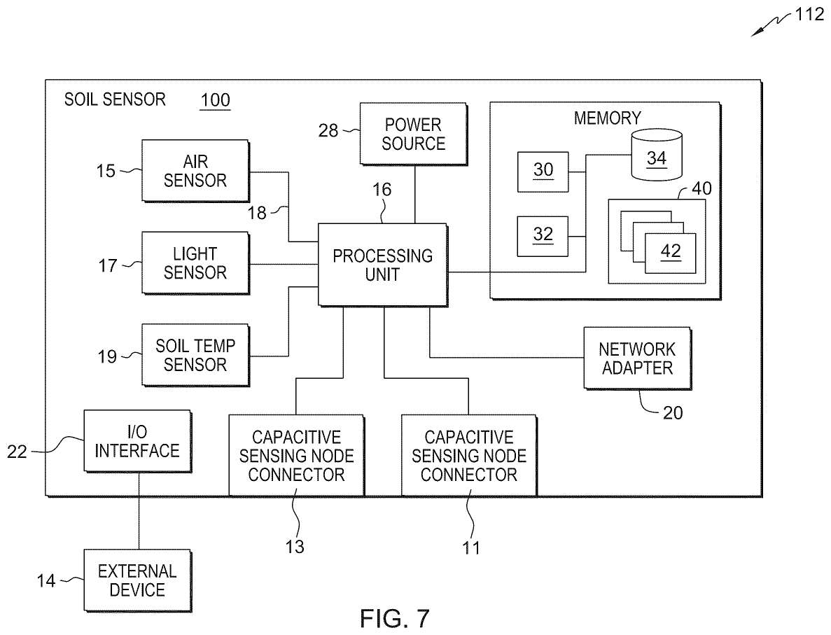

An example of the sensors 112 circuitry is shown in FIG. 7. The schematics of the circuit board provide the necessary components to collect the data from the capacitive sensing elements and to process the data. Sensor 112 as a computing node is shown in the form of a general-purpose computing device. The components of the sensor 112 may include, but are not limited to, one or more processors or processing units 16, a system memory 28, an air sensing unit 15, a light sensing unit 17, a soil temperature sensing unit 19, a power source 28, a capacitive sensing node connector 13, and capacitive sensing node connector 11, and a bus 18 that couples various system components. In some embodiment, the capacitive sensing node connector 11 and 13 may be physical connectors for the probe 400 to connect the capacitive sensing nodes 11 and 13 to the processing unit 16. In other embodiments, the capacitive sensing node connectors 11 and 13 may be integrated into the circuit board probe 400 unitary component as wires which connect the capacitive sensing nodes 11 and 13 to the processing unit 16. The capacitive sensing nodes 11 and 13 may be setup similar to the sensor in FIG. 5 or FIG. 6 as both have at least two capacitive sensing nodes, just in different structural formations but are able to function in similar manners. The processing unit 16 is capable of both receiving data as well as operating the commands to effectively measure the various properties of the soil.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

System memory 28 can include computer system readable media in the form of volatile memory, such as random-access memory (RAM) 30 and/or cache memory 32. computing device 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

The air sensing unit 15 collects the air temperature and humidity sensor to monitor ambient air conditions. In one embodiment, there are two small apertures in the housing that allow for ventilation. There is a piece of foam around the sensor and a cutout slot in the circuit board to provide thermal isolation from the rest of the product interior.

The light sensing unit 17 is a phototransistor to roughly monitor light conditions. This is only used to determine light schedules and whether the lights are on or off. In one embodiment, the phototransistor is integrated into the housing with the most light coming through. Other embodiments could include improved light monitoring for PAR (photosynthetically active radiation) measurements.

The soil temperature sensing unit 19 measures the temperature of the soil. This is contained within the housing, but as close to the soil as possible. The soil temperature is reported to the user, but its purpose is also to better compensate the soil moisture and EC measurements. Other embodiments could include temperature monitoring on the blade of the sensor for more accurate readings.

sensor 112 may also communicate with one or more computing device 104 or wearable device 114 that enable a user to interact with sensor 112; and/or any devices (e.g., network card, modem, etc.) that enable sensor 112 to communicate with one or more other computing devices. The high frequency connection and the low frequency connection permit the connection of the probe(s) to the circuit board wherein the connections electrically connect the respective capacitive sensing nodes 11 and 13 to the circuit board and physically connect the probe(s) to the circuit board. In some embodiments, the probe(s) is removable and allows for the replacement of the probe(s). This provides a benefit of creating a variety of probe(s)s with different capacitive sensing node designs to allow for the collection of various types of data. In the depicted embodiment, the circuit board and the probe(s) are a single element, where the capacitive sensing nodes 11 and 13 are integrated into the circuitry of the circuit board. Such communication can occur via Input/Output (I/O) interfaces 22. Still yet, sensor 112 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to microcode, device drivers, redundant processing units, external disk drive arrays, RAID systems, tape drives, and data archival storage systems, etc.

The program(s) described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

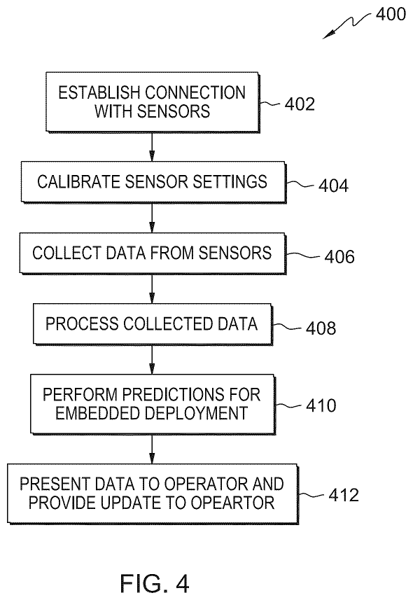

FIG. 4 depicts a flowchart 400 of the operational steps to provide the user with information they are able to user to better cultivate their crops, in accordance with one embodiment of the present invention.

In step 402, the management engine 110 establishes the connection with the sensors 112. The connection with the sensors 112 (hereinafter “sensors”) may be a manual process where a user needs to allow connection of the sensors to the management engine 110. In other embodiments, once the sensor is within range it is automatically connected to the management engine 110. The sensors have integrated hardware to wireless connect to a network, the limitation of the software determines the connection process of the sensors to the network and thus to the management engine 110.

In some embodiments, the sensors are categorized into zones or areas, based on the physical location of the sensor, the type of sensor, or the user preference. These zones create different groups that allow for further customization to the data which is collected, when it is collected, and how data is processed and presented to the user. For example, a specific plot of plants may have multiple sensors in the plot. This plot would be identified as a zone, and all the sensors in the plot can operate as a group to collect multiple data points through the plot to keep all the plants taken care of. Additionally given that different sensors may monitor different aspects or components, one zone may be all of one sensor type while another zone is a different sensor type. This is all adjustable by the user, or the management engine 110 may automatically setup the zones based on predetermined settings or restrictions.

In step 404, the management engine 110 calibrates the sensors, so that they are correctly setup for the specific environment and/or plant life to be used on/with. This allows the sensors to be able to collect the most accurate data possible based on the sensor's abilities. This may include the tuning of the circuits (or strips) within each sensor. This tuning process may involve Nordic semiconductor antenna tuning guidance or the like. In other embodiments, the circuits are tested at various frequencies until an ideal frequency is set for the operation of that specific circuit based on the specific value which is to be collected by the circuit. The adjustment of the sensors settings is based on the type of data which can be collected by each sensor. This may include setting limits to data types which are designed to trigger warnings or adjustments to exterior elements of the system. For example, if a watering system is integrated into the network, the sensors can be set with limits on the moisture values where if it drops below a predetermined value, the watering system is then activated. Provided the sensors are able to collect data related to a plurality of characteristics of the soil and the environment multiple limits may be set, and complex limits may be set involving more than one variable.

In step 406, once the system is activated, the sensors begin to collect data from the soil and the environment, and that data is transferred to the management engine 110. The sensors collect data in a raw format based on the physical components of the sensor, such as, but not limited to a capacitive sensor, a thermistor which is in contact with the soil, and an air sensor data, which each may collect data in specific formats. The raw data is based on the features of the sensor and the type of data which the sensor is able to collect. The environmental data may be related to soil volumetric water content, soil electrical conductivity value, soil temperature, air temperature, humidity value, or air vapor pressure deficit for example. Each of these may be collected in specific formats based on the mechanical/electrical components of the sensor. These different types of data which are collected by the sensors may be adjusted throughout the data collection step. For example, the frequency of the data collection by the sensor can be reset, adjusted manually, or may be limited by the sensor. In some embodiments, this involves activating and deactivating different circuits within each sensor. In specific embodiments, the data collected is based on a required parameter(s) that the user desires, such as a bulk electrical conductivity value, a volumetric water content value, an apparent dielectric permittivity value, or an ideal watering time value, that the user desires and the sensor is able to calculate the required data. These ideal values require specific measurable values which are collected by the sensors.

The parameter can also include/be optimized for an ideal range for each stage of plant life. For instance if there are seven stages of the plant's life cycle the early stages may have high temperature range and slowly drift down throughout the plant's life to increase yield. For pore EC (fertilizing) there is an ideal range to ramp up towards the end of plant life but then ramp down prior to harvest. Each sensor can be tied to a group/zone of plants with finer granularity of ideal environmental conditions over the life of the plant. This is advantageous because user s are able to greatly increase their yield by maintaining tight controls on specific parameters and optimizing for each specific stage of plant life. This practice is called “Crop Steering” by commercial cultivators. Users/operators can be notified by notifications through the computing device 104 or through the wearable device 114.

In step 408, the management engine 110 processed the collected data to convert the raw data into a readable format. In some embodiments, the data collected by the sensors is not directly readable by the management engine 110, so the gateway 103 converts the data so that the management engine 110 is able to read the data. In some embodiments, this is not needed as the management engine 110 is able to read multiple data format types. The gateway 103 is able to convert multiple different types of data with are received from the sensors to a readable format for the management engine 110.

The manipulation of the data may occur at predetermined intervals based on the requirements of the overall system and when the data is to be sent to the management engine 110. For example, the manipulation of the data may occur once daily, or may occur hourly based on the user s desired updates or notification settings. The processed data is able to be read by the receiving component(s), this allows the computing device 104 to present the data to the user, through a user interface. It also allows the processed data to be readable by the wearable device 114 to create the virtual reality environment or present the data through the augmented reality.

In step 410, the management engine 110 performs the predictions for the embedded deployment of the plant care tasks and schedules (e.g. predict plant care data). The predictive plan care calculation assists in the implementation of irrigation, fertilization, and in some aspects HVAC settings based on the plant type, the plant needs, the soil properties, and the environment properties to create an all-around ideal growing environment for the specific plant(s). In one embodiment, the management engine 110 uses data which was collected over the past two weeks (or another predetermined time period of past days) and processes this data to generate a prediction for the a future predetermined time frame (e.g. the next three days) to determine any aspects of the plant care data which will be outside the set parameters or need to be handled by the user. The prediction is related to readings of the sensors to predict what changes may occur in the soil or the environment for the user to adjust their system accordingly. The old data is used with the management engine 110 and a neural network to predict the future readings. In some embodiments, the management engine 110 is able to connect with various third party sources to collect data which is outside the ability of the sensors, for example weather forecasts and uses this data with the previously collected data to further refine the predictions and use this third party data to adjust the potential data collected by the sensor. The predictive plant care data may also include the use of machine learning models which are generated to assist with the predictive analytics. For example, if it is expected that the soil water saturation will be below the ideal value in the coming days, but a storm is expected, the management engine 110 would indicate as such to the user as a potential no issue given the weather.

In step 412, the management engine 110 presented the data to the operator and provides updates to the user. The user is alerted through the computing device 104 or wearable device 114 of any parameters which could be out of the appropriate range over future period (e.g. the next three days), so that the user is able to adjust manually or automatically the system components to keep the parameters within range, or to allow the user ample time to adjust the system when the time is right. For example, if the management engine 110 predicts that on the third day the moisture of the soil will be low, watering then would not improve that parameter. The user is able to water on the third day to keep the soil moisture within range. This predictive plan care data may be presented to a user to allow for the manual adjustment of the variables through the user interface or through the augmented or virtual reality. If the system is integrated with a watering system, HVAC system, and the like to automatically control the variables, the management engine 110 is able to control these third-party systems to make the necessary adjustments to bring the variables within the desirable ranges.

As shown in FIGS. 8 and 9, a user interface on a computing device 104 is shown. FIG. 8 depicts a user interface 800 where a notification 801 is presented to the user indicating the predictions and the updates for the system. These included are shown for exemplary purposes such as air temperature prediction 802, low soil moisture 803, high humidity 804, and high VPD 805. These predictions and updates are based on the calculations and are provided to the user at predetermined intervals, upon the calculation, or at specific events occurring. FIG. 9 depicts a user interface 900 showing the creation of the zones. The zones are groupings of sensors or systems which operate in a group. The user is able to create a new zone through icon 906 when needed. The user is able to select an interval for alerts related to the zone through an interactive icon 907. Section 902 shows a list of all the sensors and systems within the entire environment or facility. Section 901 shows a list of all the sensors and systems which are already present in the specific zone. Through icons 903 the user is able to add or remove the sensors or systems from the zone. Once the updates have been made, the user is able to apply the updates through icon 904 and is able to set threshold values for the sensors or systems through icon 905. These threshold values may be set for all specific sensor types, or systems within the zone to create a uniform alert type for all the sensors. Where the user is able to add sensors to a zone, remove sensors from a zone, switch between zones, and manipulate the zone settings of the system.

The user interface provides visual indicators of which parameters will be out of range in the future date (or future date range). In some embodiments, the information provided to the user gives a time frame in which to act or when to expect that the parameter will be outside the threshold values. For example, the illustration shows that the air temperature will be “high” within 4 hours, giving the user the ability to know when to adjust the system.

As shown in FIGS. 10 and 11, an example of virtual reality and augmented reality, respectively, is shown. FIG. 10 shows the virtual reality 1000 allows the user to take a virtual tour of the garden/facility and upon the visualization of each of the plants or zones, the related data is presented to the user, and they are able to interact with the data which is presented. The virtual reality 1000 is achieved through the wearable device. The various methods and systems the user is able to interact with the sensors and the facility to take care of the plants. The user is able to see each sensor 106A, 106B, 107A, 106C, and 107B which are each associated with one plant 1004 (or area around each plant 1004). The user is able to interact with each of the sensors, and when the user does the icons 1002, 1001, and 1003 appear in a close proximity to the respective sensor and allow the user to see the information collected from the sensor related to a specific parameter. As shown the icons have rings around them that are partially full (black and white portions). This indicates if the parameter is within the threshold and how close it is to being outside the threshold value. It is a visual indicator for the user to know if changes need to be made. The user is then able to interact with the icons 1002, 1001, and 1003 to either change the variable or receive additional information about that reading. The user is able to see more than one sensor information at a time as shown. The icons 1001, 1002, and 1003 are shown with a warning signal around the icon, and for example, as the parameter approaches a max or min value, the icon may change size, shape, color or the like to provide visual warning to the user.

As the user using the virtual reality hardware looks around the environment the various parameters and the like are presented to the user to interact with. The user is also able based on the virtual reality hardware abilities can interact with the sensor data and activate various systems (HVAC, watering, etc.) within the environment to keep the parameters within the threshold values. In some embodiments, sensors where the parameters are outside the threshold values appear in a different version to alert the user that there are issues with the sensor or the parameters, such as, but not limited to, appearing in red or with a warning sign.

FIG. 11 shows an example of the augmented reality 1100, where the user is wearing the wearable device 114 and upon looking or being with a predetermined proximity to a sensor 106A, 106B, 107A, 106C, and 107B, plant 1004, or zone, the data is populated into the user's wearable device 114 and the user is able to interact with the data and receive additional data from the management engine 110. The augmented reality 1100 is achieved through the use of the wearable device that is connected to the network. As shown window 1101 appears in the augmented reality wearable device and is associated with a specific sensor. In the depicted embodiment that is sensors 106A. This window shows icons 1102, 1103, and 1104 which are the parameter or readings by the sensor. The icons 1102, 1103, and 1104 are shown as visual indications to inform the user of the parameter approaching a max or min threshold value, and as the parameter approaches this max or min value, the readings within shapes which as the respective icon may change size, shape, color or the like to provide visual warning to the user. As shown the icons have rings around them that are partially full (black and white portions). This indicates if the parameter is within the threshold and how close it is to being outside the threshold value. It is a visual indicator for the user to know if changes need to be made. In some embodiments, sensors where the parameters are outside the threshold values appear in a different version to alert the user that there are issues with the sensor or the parameters, such as, but not limited to, appearing in red or with a warning sign.

The analysis and prediction generation happens on a substantially continuous basis so that each future day has a prediction which is most accurate based on the most recent data. This provides the user with the most current and accurate information about the next three days (or the future dates at a longer or shorter range) so that they can adjust the system to keep all the parameters within range.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein that are believed as maybe being new, this understanding, as indicated by use of the term “present invention,” is tentative and provisional and subject to change over the course of patent prosecution as relevant information is developed and as the claims are potentially amended.

The foregoing descriptions of various embodiments have been presented only for purposes of illustration and description. They are not intended to be exhaustive or to limit the present invention to the forms disclosed. Accordingly, many modifications and variations of the present invention are possible in light of the above teachings will be apparent to practitioners skilled in the art. Additionally, the above disclosure is not intended to limit the present invention. In the specification and claims the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g. attached, adhered, joined) are to be construed broadly and may include intermediate members between a connection of elements and relative movement between elements. As such, joinder references do not necessarily infer that two elements are directly connected and in fixed relation to each other. Moreover, network connection references are to be construed broadly and may include intermediate members or devices between network connections of elements. As such, network connection references do not necessarily infer that two elements are in direct communication with each other. In some instances, in methodologies directly or indirectly set forth herein, various steps and operations are described in one possible order of operation, but those skilled in the art will recognize that steps and operations may be rearranged, replaced or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method.

Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements and/or substantial equivalents.

What is claimed is:

1. A computer implemented method for predicting ideal environmental conditions to grow plants, the method comprising:
establishing, by one or more processors, a connection with at least one sensor, wherein the sensor is able to collect data related to at least one environmental condition;
grouping, by one or more processors, the at least one sensor into at least one zone;
calibrating, by the one or more processors, the at least one sensor within each zone, wherein the calibration is based on a set of environmental conditions desired for a plant and each of the at least one sensor within the zone are calibrated substantially similar;
collecting, by the one or more processors, data related to the at least one environmental condition from the at least one sensor;
manipulating, by the one or more processors, the collected data, wherein the data is manipulated from a first format to a second format, wherein the second format is readable by a predictive analyzing engine;
performing, by the one or more processors, a predictive model manipulation on the manipulated collected data;
alerting, by the one or more processors, a user with a recommendation based on the predictive model; and
manipulating, by the one or more processors, a user interface to identify specific environmental conditions to be adjusted and a predetermined adjustment of those environmental conditions.

2. The method of claim 1, further comprising, comparing, by the one or more processors, the calculated future values to the set of environmental conditions desired for the plant.

3. The method of claim 1, wherein the calculated future values are calculated for a predetermined future time frame.

4. The method of claim 1, further comprising, generating, by the one or more processors, visualizations of the collected data, wherein the visualizations are viewable through a wearable device.

5. The method of claim 4, wherein the visualizations include future values of the environmental conditions calculated by the predictive model.

6. The method of claim 5, wherein the predictive model manipulation uses data collected from third party sources to assist in the calculation of the future values of the environmental conditions.

7. The method of claim 4, wherein the visualizations are within an augmented reality environment.

8. A system for predicting ideal environmental conditions to grow plants, the system comprising:
a CPU, a computer readable memory and a computer non-transitory readable storage medium associated with a computing device;
establishing a connection with a group of sensors and at least on environmental system;
forming zones, wherein each zone contains at least one sensor;
setting a set of parameters for a first group of sensors within one of the zones;
collecting environmental data from the first group of sensors based on the set of parameters, wherein the environmental data is received by a gateway;
manipulating the collected environmental data, wherein the data is manipulated from a first format to a second format, wherein the second format is readable by a predictive analyzing engine;
performing a predictive model manipulation on the manipulated collected environmental data, wherein a future value of the environmental data is calculated;
generating visualizations of the collected environmental data and the future value of the environmental data; and
presenting the visualizations to a user's interface of a device.

9. The system of claim 8, further comprising, connecting the group of sensors and at least one environmental system to a gateway.

10. The system of claim 8, wherein the device is a wearable device capable of creating an augmented reality or a virtual reality environment.

11. The system of claim 8, further comprising, comparing the future value of the environmental data to a set of required values of the environmental data.

12. The system of claim 8, wherein the visualizations incorporate threshold values of the environmental data and the future value of the environmental data.

13. The system of claim 10, wherein the visualizations depict of the collected environmental data incorporates the group of sensors creating alerts based on the different of the future value of the environmental data to the set of required values of the environmental data, based on a predetermined value.

14. The system of claim 8, wherein the adjusting of the at least one setting of the at least one sensor is based on a set of required value of the environmental data.

15. A computer program product for predicting ideal environmental conditions to grow plants, the computer program product comprising:
a computer non-transitory readable storage medium having program instructions embodied therewith, the program instructions executable by a computing device to cause the computing device to:
program instructions to calibrate a set of sensors, wherein a frequency of at least one circuit is adjusted based on a desired environmental condition to be measured;
program instructions to collect data from the set of sensors, wherein the collected data is related to a set of variables and is provided to a gateway, and wherein the data is collected at a predetermined time frames;
program instructions to process the collected data;
program instructions to manipulate the collected data from a first format to a second readable format for a predictive engine;
program instructions to analyze the manipulated collected data, wherein the manipulated collected data is analyzed to determine future values a set of parameters based on a set of threshold values; and
program instructions to generate visualizations of the collected data and the future values.

16. The computer program product of claim 15, further comprising, program instructions to compare the future values to a set of base values.

17. The computer program product of claim 15, further comprising, program instructions to further analyze the manipulated collected data with a set of third-party data to calculate a second set of future values for the set of variables.

18. The computer program product of claim 16, further comprising, program instructions to generate alerts on a user device based on the difference between the future values and the set of base values.

19. The computer program product of claim 15, further comprising generating a virtual reality environment wherein the visualizations are incorporated into the virtual reality environment.

20. The computer program product of claim 15, wherein the visualizations of the collected data and the future values is presented to the user within augmented reality hardware wherein a proximity of the collected data or the future values to a threshold value is emphasized within the visualizations.
